(19) United States
(12) Patent Application Publication
(10) Pub. No.: US 2010/0041668 A1
(43) Pub. Date: Feb. 18, 2010

(54) Compositions and methods for treating thrombocytopenia

(75) Inventors: Ken-Ichi Suzuki, Tokyo (JP); Keizo Sugasawa, Tokyo (JP)

Correspondence Address: Davidson, Davidson & Kappel, LLC, 485 7th Avenue, 14th Floor, New York, NY, 10018, US

(73) Assignee: Astellas Pharma Inc., Tokyo (JP)

(21) Appl. No.: 12/583,359

(22) Filed: Aug. 19, 2009

Related U.S. Application Data
(63) Continuation of application No. 11/593,758, filed on Nov. 7, 2006.
(60) Provisional application No. 60/734,426, filed on Nov. 8, 2005.

(51) Int. Cl. A61K 31/496; A61P 7/00

(52) U.S. Cl. 514/253.1

(57) ABSTRACT

The present invention in certain embodiments is directed to a pharmaceutical dosage form comprising a therapeutically effective amount of a first agent that agonizes a human TPO receptor by binding to the rhTPO binding site of the human TPO receptor; and a therapeutically effective amount of a second agent that agonizes the human TPO receptor by binding to a binding site of the human TPO receptor distinct from the rhTPO binding site.

This application claims priority to U.S. Provisional Application No. 60/734,426, filed Nov. 8, 2005, the contents of which are hereby incorporated by reference in its entirety.

### FIELD OF THE INVENTION

The present invention is directed to dosage forms and methods of treating or preventing thrombocytopenia with thrombopoietin receptor agonists.

### BACKGROUND OF THE INVENTION

A platelet is an anuclear blood cell playing an important role in physiological hemostasis and pathological thrombosis, and is continuously produced from megakaryocytes in a living organism. The platelet originates from pluripotent stem cells like other blood cells. Specifically, the pluripotent stem cell becomes a megakaryocytic progenitor cell, from which megakaryoblasts, promegakaryocytes, and megakaryocytes are formed. During the maturation of a megakaryocyte, premature megakaryocytes only carry out DNA synthesis without involving cell division to become a polyploid. Thereafter, cytoplasm begins to mature to form a platelet separation membrane, and a platelet is released by cytoplasm fragmentation.

Decreases in the number of platelets due to various hematopoietic dysfunctions, such as myelodysplastic syndrome, or chemotherapy or radiotherapy for malignant tumors and the like, cause serious symptoms such as hemorrhagic tendencies. There have been many attempts at developing various drug and non-drug therapies for increasing the number of platelets for the purpose of treating such dysfunctions. One such therapy is platelet transfusion. Although platelet transfusion has become a useful means for treating thrombocytopenia, a sufficient amount of platelets cannot be provided, and it is difficult to sufficiently improve thrombocytopenia because of a short life span of transfused platelets and the like. Additionally, platelet transfusions involve problems including viral infection, production of all antibodies, Graft Versus Host Disease (GVHD), and the like. Thus, there is a demand for the development of a medicament for mitigating hematopoietic suppression caused by various conditions or therapies to thereby promote the recovery of platelet numbers.

It has been reported that thrombopoietin (hereinafter referred to as “TPO”), which is a c-Mp1 ligand playing an important role in differentiation into megakaryocytes, has been cloned, and that it stimulates differentiation and proliferation of megakaryocytes to promote production of platelets (Kaushansky K. et al., Nature, 369, 568-571, 1994). Clinical tests on TPO as an agent for increasing the number of platelets have been carried out, and its availability and admissibility in humans have been confirmed. However, because a neutralizing antibody was confirmed in a clinic test of PEG-rHuMGDF, a kind of TPO (163 N-terminal amino acids of native TPO modified with polyethyleneglycol) (Li J. et. al., Blood, 98, 3241-3248, 2001, and Basser R. L. et. al., Blood, 99, 2599-2602, 2002), there is a concern about immunogenicity of TPO. Furthermore, because TPO is a protein, it is decomposed in the digestive tract, and thus is not practical as an agent for oral administration. For the same reason, it is considered that a low molecular peptide is also not practical as an agent for oral administration. Under these circumstances, work in developing a nonpeptide c-Mpl ligand, which has low immunogenicity and can be orally administrated, for the purpose of treatment of thrombocytopenia, has been conducted.

For example, benzazepine derivatives are disclosed in Japanese Laid-Open Patent Publication No. Hei 11-152276. Acylhydrazone derivatives are described in WO 99/11262. Diazonaphthalene derivatives are described in WO 00/35446. Pyrrolocarbazole derivatives are described in WO 98/09967. Pyrrolophenanthridine derivatives are described in Japanese Laid-Open Patent Publication No. Hei 10-212289, and pyrrolophthalimide derivatives are described in Japanese Laid-Open Patent Publication No. 2000-44562.

Furthermore, WO 01/07423 describes a compound represented by the following general Formula (VII) having an activity of increasing the number of platelets:

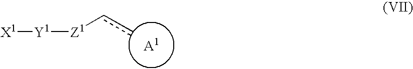

(wherein symbols are as defined in the publication)

WO 01/53267 describes a compound represented by the following general Formula (VIII) which has an activity of increasing the number of platelets:

X₁—Y₁—Z₁—W₁ (VIII)

(wherein symbols are as defined in the publication).

Japanese Patent Publication No. 3199451 describes that a 2-acylaminothiazole compound has the effects of a cholecystokinin and gastrin receptor agonist. The Chemical and Pharmaceutical Bulletin, 25, 9, 2292-2299, 1977 describes that a 2-acylaminothiazole compound has anti-inflammatory effects. However, there is no description about activity for increasing the number of platelets.

WO 03/062233 and EP 1466912 A1 disclose 2-acylaminothiazole derivatives which have effect on the proliferation of human c-Mpl-Ba/F3 cells and an activity of increasing platelets based on the effect of promoting the formation of megakaryocytic colonies.

WO 04/029049 discloses the maleic salt of a 2-acylaminothiazole derivative.

Accordingly, there exists a need in the art for compositions and methods of treating thrombocytopenia.

All of the references cited herein, including the foregoing, are hereby incorporated by reference in their entireties for all purposes.

### SUMMARY OF THE INVENTION

It is an object of certain embodiments of the invention to provide pharmaceutical compositions of TPO receptor agonists for the treatment of thrombocytopenia.

It is an object of certain embodiments of the invention to provide methods of treating thrombocytopenia utilizing TPO receptor agonists.

In view of the above objects, and others, in certain embodiments, the present invention is directed to a pharmaceutical dosage form comprising a therapeutically effective amount of a first agent that agonizes a human TPO receptor by binding to the rhTPO binding site; and a therapeutically effective amount of a second agent that agonizes a human TPO receptor at a binding site distinct from the rhTPO binding site.

In certain embodiments, the invention is directed to a method of treating thrombocytopenia comprising co-administering to a patient in need thereof, a therapeutically effective amount of a first agent that agonizes a human TPO receptor by binding to the rhTPO binding site and a therapeutically effective amount of a second agent that agonizes the human TPO receptor at a binding site distinct from the rhTPO binding site.

In certain embodiments, the present invention is directed to a method of treating thrombocytopenia comprising administering an effective amount of a compound that agonizes a human TPO receptor by binding to a binding site of the human TPO receptor distinct from the rhTPO binding site to increase platelets at least about 150%, at least about 200%, at least about 270%, at least about 300%, at least about 1,000% or at least about 5,000%.

In certain embodiments, the present invention is directed to a method of treating thrombocytopenia by administering a therapeutically effective amount of a human TPO receptor agonist to a patient such that administration of the agonist increases platelets up to about 300%, up to about 500%, up to about 1,000%, up to about 5,000% or up to about 10,000%.

In certain embodiments, the present invention is directed to a method of treating thrombocytopenia, comprising administering to a patient already receiving treatment for the thrombocytopenia a therapeutically effective amount of a compound that agonizes a human TPO receptor by binding to a binding site of the human TPO receptor distinct from the rhTPO binding site.

In certain embodiments, the present invention is directed to a method of treating thrombocytopenia, comprising administering to a patient in need thereof a dose of at least 0.01 mg/kg/day of a compound that agonizes a human TPO receptor by binding to a binding site of the human TPO receptor distinct from the rhTPO binding site; determining the platelet count in the patient after administration; and optionally adjusting the dose of the compound.

In certain embodiments, the present invention is directed to a method of treating thrombocytopenia comprising co-administering to a patient in need thereof, a therapeutically effective amount of a first agent that agonizes the human TPO receptor by binding to an rhTPO binding site and a therapeutically effective amount of a second agent that agonizes the human TPO receptor by binding to a binding site of the human TPO receptor distinct from the rhTPO binding site, wherein the second agent does not displace the first agent.

In certain embodiments, the present invention is directed to a method of treating thrombocytopenia in a patient in need of a transfusion by co-administering a therapeutically effective amount of a compound that agonizes a human TPO receptor by binding to a binding site of the human TPO receptor distinct from the rhTPO binding site and a transfusate, such that administration of the agonist increases platelets as compared to administration of the transfusate alone.

In certain embodiments, the present invention is directed to a method of treating thrombocytopenia and decreasing the incidence of viral infection and antibodies associated with a transfusion by administering to a patient in need thereof a therapeutically effective amount of a compound that agonizes a human TPO receptor by binding to a binding site of the human TPO receptor distinct from the rhTPO binding site.

In certain embodiments, the present invention is directed to a method of treating thrombocytopenia by administering to a human or animal in need thereof a dose of at least 0.01 mg/kg/day of a compound that agonizes a human TPO receptor by binding to a binding site of the human TPO receptor distinct from the rhTPO binding site, monitoring the increase in platelets produced; and adjusting the dose of the compound in order to ascertain whether an adjustment of the dose is necessary.

In certain embodiments, the present invention is directed to a method for increasing thrombocytes in a patient by administering to a patient in need thereof a therapeutically effective amount of a compound that agonizes a human TPO receptor by binding to a binding site of the human TPO receptor distinct from the rhTPO binding site.

In certain embodiments, the present invention is directed to a method of treating thrombocytopenia by diagnosing a patient in need of agonism of a human TPO receptor at a binding site distinct from the rhTPO binding site and administering to the patient a therapeutically effective amount of a compound that agonizes the human TPO receptor by binding to a binding site of the human TPO receptor distinct from the rhTPO binding site.

In certain embodiments, the present invention is directed to a method of treating thrombocytopenia by screening for a compound that agonizes a human TPO receptor by binding to a binding site of the human TPO receptor distinct from the rhTPO binding site, and administering a therapeutically effective amount of an agent to a patient in need thereof to provide an increase in thrombocytes.

In embodiments of the present invention, the TPO agonist is administered in an amount from about 0.01 mg/kg/day to about 10 mg/kg/day; from about 0.01 mg/kg/day to about 3 mg/kg/day; from about 0.5 mg/kg/day to about 3 mg/kg/day; from about 0.1 mg/kg/day to about 2 mg/kg/day or from about 1 mg/kg/day to about 3 mg/kg/day. Preferably, the TPO agonist dosed in this amount is the compound of formula X, as disclosed herein.

In certain embodiments of the present invention, the TPO agonist is administered in an amount from about 1 mg/day to about 50 mg/day; from about 5 mg/kg/day to about 30 mg/day; from about 10 mg/day to about 25 mg/day; or from about 15 mg/day to about 20 mg/day.

In preferred embodiments, the TPO agonist is administered orally.

In certain embodiments, the present invention is directed to a method of conducting a pharmaceutical business comprising screening for a compound that agonizes a human TPO receptor at a binding site of the human TPO receptor distinct from the rhTPO binding site; and selling the compound in a distribution network.

In certain embodiments, the present invention is directed to a method of conducting a pharmaceutical business comprising screening for a compound that agonizes a human TPO receptor by binding to a binding site of the human TPO receptor distinct from the rhTPO binding site; and inservicing health professionals that the compound increases thrombocytes.

In certain embodiments disclosed herein, the TPO receptor agonist is a compound that agonizes a human TPO receptor by binding to a binding site of the human TPO receptor distinct from the rhTPO binding site.

In certain embodiments disclosed herein, the TPO receptor agonist is a 2-acylaminothiazole compound of Formula (I) or a pharmaceutically acceptable salt, base, polymorph, metabolite or derivative thereof:

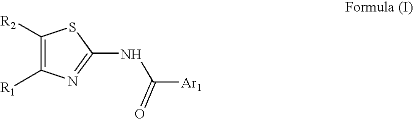

wherein Ar₁ is aryl, monocyclic aromatic heterocycle, or bicyclic condensed heterocycle, each of which may be substituted (with the proviso that when R₁ is aryl or pyridyl, each of which may be substituted with one or more groups selected from the group consisting of lower alkyl, —CO-lower alkyl, —COO-lower alkyl, —OH, —O-lower alkyl, —OCO-lower alkyl, and halogen atom, and R₂ is a group represented by the following general Formula (II); Ar₁ is not phenyl or pyridyl, each of which may be substituted with one or more groups selected from the group consisting of lower alkyl, —CO-lower alky, —COO-lower alkyl, —OH, —O-lower alkyl, —OCO-lower alkyl, and halogen atom); R₁ is aryl or monocyclic aromatic heterocycle, each of which may be substituted; R₂ is a group represented by the following general Formula (II), (III) or (IV):

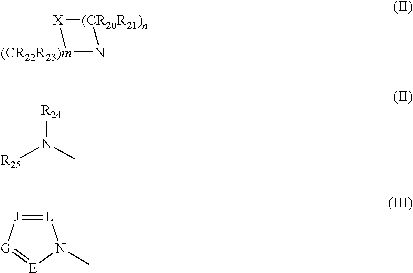

wherein n is an integer of 1 to 3; m is an integer of 1 to 3, (when n or m is an integer of 2 or more, CR₂₀R₂₁ and CR₂₂R₂₃ may be identical or different); X is O, S, or a group represented by N—R₂₆ or C(—R₂₇)—R₂₈; E, G, J, L are independently N or a group represented by C—R₂₉, with the proviso that at least one of them is C—R₂₉, R₂₀, R₂₁, R₂₂, R₂₃, R₂₆, R₂₇, R₂₈, R₂₉: which may be identical or different —H; —OH; —O-lower alkyl; optionally substituted lower alkyl; optionally substituted cycloalkyl; optionally substituted aryl; optionally substituted arylalkyl; optionally substituted aromatic heterocycle; optionally substituted aromatic heterocyclic alkyl; optionally substituted nonaromatic heterocycle; optionally substituted lower alkenyl; optionally substituted lower alkylidene; —COOH; —COO-lower alkyl; —COO-lower alkenyl; —COO-lower alkylene-aryl; —COO-lower alkylene-aromatic heterocycle; carbamoyl or amino, each of which may be substituted with one or more groups selected from the group consisting of lower alkyl and cycloalkyl, each of which may be substituted with halogen, —OH, —O-lower alkyl, or —O-aryl; —NHCO-lower alkyl; or oxo; R₂₄, R₂₅ which may be identical or different, —H, optionally substituted lower alkyl, optionally substituted cycloalkyl, or optionally substituted nonaromatic heterocycle.

In certain embodiments of the present invention, Ar₁ in the compound represented by the general Formula (I) is preferably phenyl or monocyclic aromatic heterocycle, each of which may be substituted. In certain other embodiments, Ar₁ is preferably, phenyl or pyridyl, each of which may be substituted. In other embodiments, Ar₁ is preferably phenyl which is unsubstituted at 2- and 6-positions, substituted with —H, —F, —Cl or —Br at 3-position, substituted with —F, —Cl— or —Br at 5-position, and substituted at 4-position, or pyridin-3-yl which is unsubstituted at 2- and 4-positions, substituted with —F, —Cl, or —Br at 5 position, and substituted at 6-position. In certain other embodiments, Ar₁ is preferably phenyl which is substituted at 4 position with a group consisting of —O—R_{Y}, —NH—R_{Y}, optionally substituted piperidin-1-yl and optionally substituted piperazin-1-yl, or pyridin-3-yl which is substituted at 6-position with a group consisting of —O—R_{Y}, —NH—R_{Y}, optionally substituted piperidin-1-yl, and optionally substituted piperazin-1-yl.

The “R_{Y}” is lower alkyl which may be substituted with one or more groups selected from the group consisting of —OH, —O-lower alkyl, amino which may be substituted with one or two lower alkyl, —CO₂H, —CO₂-lower alkyl, carbamoyl which may be substituted with one or two lower alkyl, cyano, aryl, aromatic heterocycle, nonaromatic heterocycle, and halogen atom.

R₁ in the compound of the general Formula (I) is preferably phenyl or thienyl, each of which may be substituted. In certain other embodiments, R₁ is preferably phenyl or thienyl, each of which may be substituted with one or more groups selected from the group consisting of halogen atoms and trifluoromethyl. In certain other embodiments, R₁ is preferably phenyl or thienyl, each of which is substituted with 1 to 3 halogen atoms (when substituted with 2 or 3 halogen atoms, the halogen atoms may be identical or different).

R₂ in the compound of the general Formula (I) is preferably a group represented by the general Formula (II). In other embodiments, R₂ is preferably a group represented by the general Formula (II) wherein n is 2, m is 2, and X is a group represented by N—R₂₆ or C(—R₂₇)—R₂₈. In certain other embodiments, R₂ is preferably 4-(piperidin-1-yl)piperidin-1-yl, 4-propylpiperidin-1-yl, 4-cyclohexylpiperazin-1-yl, or 4-propylpiperazin-1-yl.

In certain other embodiments, the present invention is directed to a compound of formula (I) above, wherein R₁ is phenyl or thienyl, each of which may be substituted with 1 to 3 halogen atoms (when substituted with 2 or 3 halogen atoms, the halogen atoms may be identical or different); R₂ is a group represented by the general Formula (II), (wherein n is 2, m is 2, and X is a group represented by N—R₂₆ or C(—R₂₇)—R₂₈); and Ar₁ is phenyl or pyridyl, each of which may be substituted.

In certain embodiments disclosed herein, the TPO receptor agonist is a 2-acylaminothiazole compound of Formula (V) or a pharmaceutically acceptable salt, base, polymorph, metabolite or derivative thereof:

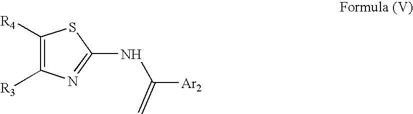

wherein Ar₂ is a group represented by Ar₁ as described in (1), with the proviso that indol-2-yl is excluded; R₃ is a group represented by R₁ as described in (1); R₄ is a group represented by R₂ as described in (1), with the proviso that a group represented by the general Formula (IV) is excluded.

In certain embodiments, Ar₂ in the compound of the general Formula (V) is preferably phenyl or monocyclic aromatic heterocycle, each of which may be substituted. In certain embodiments, Ar₂ is preferably phenyl or pyridyl, each of which may be substituted. In yet another embodiment, Ar₂ is preferably phenyl which is unsubstituted at 2- and 6-positions, substituted with —H, —F, —Cl, or —Br at 3-position, substituted with —F, —Cl, or —Br at 5-position, and substituted at 4-position, or pyridin-3-yl which is unsubstituted at 2- and 4-positions, substituted with —F, —Cl, or —Br at 5-position, and substituted at 6-position. In certain other embodiments, Ar₂ is preferably phenyl substituted at 4-position with a substituent group selected from the group consisting of —O—R_{Y}, —NH—R_{Y}, optionally substituted piperidin-1-yl and optionally substituted piperazin-1-yl, or pyridin-3-yl which is substituted at 6-position with a substituent group selected from the group consisting of —O—R_{Y}, —NH—R_{Y}, optionally substituted piperidin-1-yl and optionally substituted piperazin-1-yl.

In certain embodiments, R₃ in the compound of the general Formula (V) is preferably phenyl or thienyl, each of which may be substituted. In certain embodiments, R₃ is preferably phenyl or thienyl, each of which may be substituted with one or more groups selected from the group consisting of halogen atom and trifluoromethyl. In certain other embodiments, R₃ is preferably phenyl or thienyl, each of which is substituted with 1 to 3 halogen atoms (when substituted with 2 or 3 halogen atoms, the halogen atom may be identical or different).

In certain embodiments, R₄ in the compound of the general Formula (V) is preferably a group represented by the general Formula (II). In certain embodiments, R₄ is more preferably a group represented by the general Formula (II) wherein n is 2, m is 2, and X is N—R₂₆ or C—(R₂₇)—R₂₈. In certain other embodiments, R₄ is more preferably 4-(piperidin-1-yl)piperidin-1-yl, 4-propylpiperidin-1-yl, 4-cyclohexylpiperazin-1-yl, or 4-propylpiperazin-1-yl.

In certain embodiments, the present invention utilizes a compound of Formula (V), wherein Ar₂ is phenyl or monocyclic aromatic heterocycle, each of which may be substituted.

In another embodiment, the present invention utilizes a compound of Formula (V) wherein R₃ is phenyl or thienyl, each or which may be substituted; R₄ is a group represented by the general Formula (II); Ar₂ is phenyl or pyridyl, each of which may be substituted; n is 2, m is 2, and X is a group represented by N—R₂₆ or C(—R₂₇)—R₂₈.

In yet another embodiment, the present invention utilizes a compound of Formula (V); wherein R₃ is phenyl or thienyl, each of which is substituted with 1 to 3 halogen atoms (when substituted with 2 or 3 halogen atoms, the halogen atoms may be identical or different.); n is 2, m is 2, and X is a group represented by N—R₂₆ or C(—R₂₇)—R₂₈; R₄ is a group represented by the general Formula (II); Ar₂ is phenyl or pyridyl, each of which may be substituted; n is 2, m is 2, and X is a group represented by N—R₂₆ or C(—R₂₇)—R₂₈.

In yet another embodiment, the present invention utilizes a compound of Formula (V), wherein R₄ is 4-(piperidin-1-yl)piperidin-1-yl, 4-propylpiperidin-1-yl, 4-cyclohexylpiperazin-1-yl, or 4-propylpiperazin-1-yl; R₃ is phenyl or thienyl, each of which is substituted with 1 to 3 halogen atoms (when substituted with 2 or 3 halogen atoms, the halogen atoms may be identical or different.); n is 2, m is 2, and X is a group represented by N—R₂₆ or C(—R₂₇)—R₂₈; R₄ is a group represented by the general Formula (II); Ar₂ is phenyl or pyridyl, each of which may be substituted; n is 2, m is 2, and X is a group represented by N—R₂₆ or C(—R₂₇)—R₂₈.

In yet another embodiment, the present invention utilizes a compound of Formula (V), wherein, Ar₂ is phenyl which is unsubstituted at 2- and 6-positions, substituted with —H, —F, —Cl, or —Br at 3 position, substituted with —F, —Cl, or —Br at 5-position, and substituted at 4-position; or pyridin-3-yl which is unsubstituted at 2- and 4-positions, substituted with —F, —Cl, or —Br at 5-position, and substituted at 6-position; R₄ is 4-(piperidin-1-yl)piperidin-1-yl, 4-propylpiperidin-1-yl, 4-cyclohexylpiperazin-1-yl, or 4-propylpiperazin-1-yl; R₃ is phenyl or thienyl, each of which is substituted with 1 to 3 halogen atoms (when substituted with 2 or 3 halogen atoms, the halogen atoms may be identical or different.); n is 2, m is 2, and X is a group represented by N—R₂₆ or C(—R₂₇)—R₂₈; R₄ is a group represented by the general Formula (II); n is 2, m is 2, and X is a group represented by N—R₂₆ or C(—R₂₇)—R₂₈.

In yet another embodiment, the present invention utilizes a compound of Formula (V), wherein, Ar₂ is phenyl which is substituted at 4-position with a group selected from the group consisting of —O—R_{Y}, —NH—R_{Y}, optionally substituted piperidin-1-yl and optionally substituted piperazin-1-yl; or pyridin-3-yl which is substituted at 6-position with a group selected from the group consisting of —O—R_{Y}, —NH—R_{Y}, optionally substituted piperidin-1-yl and optionally substituted piperazin-1-yl (wherein R_{Y} is lower alkyl which may be substituted with one or more groups selected from the group consisting of —OH, —O-lower alkyl, amino which may be substituted with one or two lower alkyl, —CO₂H, —CO-lower alkyl, carbamoyl which may be substituted with one or two lower alkyl, cyano, aryl, aromatic heterocycle, nonaromatic heterocycle, and halogen atoms); R₄ is 4-(piperidin-1-yl)piperidin-1-yl, 4-propylpiperidin-1-yl, 4-cyclohexylpiperazin-1-yl, or 4-propylpiperazin-1-yl; R₃ is phenyl or thienyl, each of which is substituted with 1 to 3 halogen atoms (when substituted with 2 or 3 halogen atoms, the halogen atoms may be identical or different.); n is 2, m is 2, and X is a group represented by N—R₂₆ or C(—R₂₇)—R₂₈; R₄ is a group represented by the general Formula (II); n is 2, m is 2, and X is a group represented by N—R₂₆ or C(—R₂₇)—R₂₈.

In order that the invention described herein may be more fully understood, the following definitions are provided for the purposes of this disclosure:

By “controlled-release” it is meant for purposes of the present invention that the active agent(s) or a pharmaceutically acceptable free base, salts, polymorphs, derivatives or combinations thereof are released from the formulation at a controlled rate such that therapeutically beneficial blood levels (at least minimally effective levels and below toxic levels) of the active agent(s) are maintained over an extended period of time, e.g., for about a 8 to about 24 hours, such that the formulations are suitable for thrice, twice, or once a day administration.

The term “human patient” is meant for purposes of the present invention to be an individual who suffers from an illness relevant to the medication being administered.

By co-administration it is meant either the administration of a single composition containing both the first agent and the second agent as disclosed herein, or the administration of the first agent and the second agent as disclosed herein as separate compositions wherein at least a portion of each dosing interval overlaps.

### BRIEF DESCRIPTION OF THE DRAWINGS

FIG. 1 is a graph that depicts the number of megakaryocytes produced after G-CSF-mobilized human peripheral blood CD34⁺ cells were cultured for 14 days in serum-free liquid medium in the presence of i) FORMULA X, ii) TPO, or (iii) TPO+FORMULA X.

FIGS. 2 and 3 are graphs depicting the number of hematopoietic progenitor cells (FIG. 2) and megakaryocytic progenitor cells (FIG. 3) produced after G-CSF-mobilized human peripheral blood CD34⁺ cells were cultured for 14 days in serum-free liquid medium in the presence of i) FORMULA X, ii) TPO, or (iii) TPO+FORMULA X.

FIGS. 4, 5, and 6 are graphs depicting the time course for hematopoietic progenitor cells (FIG. 4), megakaryocytic progenitor cells (FIG. 5) and megakaryocytes (FIG. 6) after G-CSF-mobilized human peripheral blood CD34⁺ cells were cultured for 14 days in serum-free liquid medium in the presence of i) FORMULA X, ii) TPO, or (iii) TPO+FORMULA X.

### DETAILED DESCRIPTION OF THE INVENTION

In certain embodiments, the compounds utilized in the present invention are 2-acylaminothiazole derivatives structurally characterized in that an acylamino group is substituted at the 2-position thereof and that a nitrogen atom of a nitrogen-containing heterocycle is directly bound to the 5-position thereof. Such compounds are disclosed in WO 03/062233 and EP 1466912 A1, hereby incorporated by reference.

In certain embodiments, the compounds of the present invention may provide for an increase in platelets. These compounds, also known as “c-Mp1 ligands”, act by proliferating human c-Mp1 Ba/F3 cells and promoting differentiation of human CD34⁺ into megakaryocytes resulting in an increase in platelets. Proliferation of these cells may be the result of the compounds ability to bind to human thrombopoietin receptors (hereinafter “TPO receptors”).

Platelets are anuclear blood cells that play an important role in physiological hemostasis and pathological thrombosis, and are continuously produced from megakaryocytes in a living organism. Platelets originate from pluripotent stem cells like other blood cells. Specifically, the pluripotent stem cells become a megakaryocytic progenitor cell, from which megakaryoblasts, promegakaryocytes, and megakaryocytes are formed. During maturation of megakaryocytes, premature megakaryocytes only carry out DNA synthesis without involving cell division to become a polyploid. Thereafter, cytoplasm begins to mature to form a platelet separation membrane, and a platelet is released by cytoplasm fragmentation.

As a result of the ability of the present compounds to increase platelets, the compositions and methods described herein may be useful for the treatment or prevention of thrombocytopenia caused by a pre-existing condition, such as, but not limited to AIDS, advanced liver disease, myelodysplastic syndrome, or thrombocytopenia caused by an existing or previously administered drug therapy. The thrombocytopenia can also be caused by idiopathic thrombocytopenic purpura or disease inherent thrombocytopenia.

Thrombocytopenia is a disorder in which the number of platelets is abnormally low. Thrombocytopenia is sometimes associated with abnormal bleeding, drug therapy (e.g., chemotherapy), and even many medical disorders. Signs and symptoms generally include difficulty in clotting, nose bleeds and bruising.

Current treatments for thrombocytopenia include, but are not limited to, blood transfusions, oral corticosteroids, immunosupressants, spleen removal or treatment of the underlying condition.

In certain other embodiments, the compounds described herein may be administered with additional active agents that are useful for treating thrombocytopenia and/or other blood disorders. In certain embodiments, the agonist is co-administered with another hematopoietic growth factor, such as erythropoietin, stem cell factor, Interleukin (Interleukin 1-12), granulocyte/macrophage colony-stimulating factor (GM-CSF), granulocyte colony-stimulating factor (G-CSF), monocyte/macrophage colony-stimulating factor (M-CSF or CSF-1), macrophage colony-stimulating factor (M-CSF), thrombopoietin (TPO) or any combinations and mixtures thereof.

In certain other embodiments, the compounds described herein may be co-administered with other experimental or investigational hematopoietic factors, such as, but not limited to rThrombopoietin (in development for CIT/Pliva), AMG-531 (Phase II for ITP/Amgen) and GSK-115 (Phase II for Glaxo-Smithkline).

In yet another embodiment of the present invention, the compounds described herein may be co-administered with many other pharmaceutical agents. For example, the compounds described herein may be co-administered with analgesic agents, antihemophilic factor, antihemorrhagic agents, antineoplastic agents, antiulcerative agents, antacids, corticosteroids, growth hormones, hematinic agents, immunosupressants, platelet-activating factors and any combinations and mixtures thereof.

In certain embodiments, the compounds utilized in the present invention include, but are not limited to:
N-[4-(4-chlorothiophen-2-yl)-5-(4-cyclohexylpiperazin-1-yl)thiazol-2-yl]-3-fluoro-4-hydroxybenzamide,
3-chloro-N-[4-(4-chlorothiophen-2-yl)-5-(4-cyclohexylpiperazin-1-yl-)thiazol-2-yl]-4-(2-hydroxyethoxy)benzamide,
N-[4-(4-chlorothiophen-2-yl)-5-(4-propylpiperidino)thiazol-2-yl]-2-methoxyisonicotinamide,
N-[4-(4-chlorothiophen-2-yl)-5-(4-cyclohexylpiperazin-1-yl)thiazol-2-yl]isoquinoline-6-carboxamide,
3-chloro-N-[4-(4-chlorothiophen-2-yl)-5-(4-propylpiperazin-1-yl)thi-azol-2-yl]-4-(2-hydroxyethoxy)benzamide,
5-chloro-N-[4-(4-chlorothiophen-2-yl)-5-(4-cyclohexylpiperazin-1-yl-)thiazol-2-yl]-6-(3-hydroxypropoxy)nicotinamide,
5-chloro-N-[4-(4-chlorothiophen-2-yl)-5-(4-cyclohexylpiperazin-1-yl-)thiazol-2-yl]-6-[(3-hydroxypropyl)amino)]nicotinamide,
1-(3-chloro-5-{[4-(4-chlorothiophen-2-yl)-5-(4-cyclohexylpiperazin-1-yl-)thiazol-2-yl]carbamoyl}-2-pyridyl)piperidine-4-carboxylic acid,
1-(3-chloro-5-{[4-(4-chlorothiophen-2-yl)-5-(4-propylpiperazin-1-yl-)thiazol-2-yl]carbamoyl}-2-pyridyl)piperidine-4-carboxylic acid,
N-[4-(4-chlorothiophen-2-yl)-5-(4-cyclohexylpiperazin-1-yl)thiazol-2-yl]-4-(4-cyanopiperidino)-3,5-difluorobenzamide,
1-(2-chloro-4-{[4-(4-chlorothiophen-2-yl)-5-(4-cyclohexylpiperazin-1-yl-)thiazol-2-yl]carbamoyl}phenyl)piperidine-4-carboxylic acid,
1-(2-chloro-4-{[4-(4-chlorothiophen-2-yl)-5-(4-cyclohexylpiperazin-1-y-l)thiazol-2-yl]carbamoyl}-6-fluorophenyl)piperidine-4-carboxylic acid,
1-(2-chloro-4-{[4-(4-chlorothiophen-2-yl)-5-(4-propylpiperazin-1-yl-)thiazol-2-yl]carbamoyl}phenyl)piperidine-4-carboxamide,
5-chloro-N-[4-(4-chlorothiophen-2-yl)-5-(4-cyclohexylpiperazin-1-yl-)thiazol-2-yl]-6-(4-hydroxymethylpiperidino)nicotinamide,
1-(3-chloro-5-{[5-(4-cyclohexylpiperazin-1-yl)-4-(4-fluorophenyl-)thiazol-2-yl]carbamoyl}-2-pyridyl)piperidine-4-carboxylic acid,
1-(3-chloro-5-{[5-(4-cyclohexylpiperazin-1-yl)-4-(3-trifluoromethyl-phenyl)thiazol-2-yl]carbamoyl}-2-pridyl)piperidine-4-carboxylic acid,
5-chloro-N-[4-(4-chlorothiophen-2-yl)-5-(4-cyclohexylpiperazin-1-yl-)thiazol-2-yl]-6-{4-[(2-methoxyethyl)carbamoyl]piperidino}nicotinamide,
5-chloro-N-[4-(4-chlorothiophen-2-yl)-5-(4-cyclohexylpiperazin-1-yl-)thiazol-2-yl]-6-{4-[(3-methoxypropyl)carbamoyl]piperidino}nicotinamide,
5-chloro-N-[4-(4-chlorothiophen-2-yl)-5-(4-cyclohexylpiperazin-1-yl-)thiazol-2-yl]-6-[4-(morpholinocarbonyl)piperidino]nicotinamide, and
pharmaceutically acceptable salts thereof.

In certain other embodiments, the compounds utilized in the present invention include, but are not limited to:
N-[4-(4-chlorothiophen-2-yl)-5-(4-cyclohexylpiperazin-1-yl)thiazol-2-yl]-2-methoxyisonicotinamide,
3-chloro-N-[4-(4-chlorothiophen-2-yl)-5-(4-cyclohexylpiperazin-1-yl-)thiazol-2-yl]-4-(2-methoxyethoxy)benzamide,
N-[4-(4-chlorothiophen-2-yl)-5-(4-cyclohexylpiperazin-1-yl)thiazol-2-yl-]quinoline-6-carboxamide,
3-chloro-N-[4-(5-chlorothiophen-2-yl)-5-(4-cyclohxylpiperazin-1-yl)-thiazol-2-yl]-4-(2-hydrxyethoxy)benzamide,
3-chloro-N-[4-(4-chlorothiophen-2-yl)-5-(4-cyclohexylpiperazin-1-yl-)thiazol-2-yl]-5-fluoro-4-(2-hydroxyethoxy)benzamide,
3-chloro-N-[4-(4-chlorothiophen-2-yl)-5-(4-cyclohexylpiperazin-1-yl-)thiazol-2-yl]-4-(3-hydroxypropoxy)benzamide,
3,5-dichloro-N-[4-(4-chlorothiophen-2-yl)-5-(4-cyclohexypiperazin-1-yl)thiazol-2-yl]-4-(2-hydroxyethoxy)benzamide,
3-bromo-N-[4-(4-chlorothiophen-2-yl)-5-(4-cyclohexylpiperazin-1-yl)-thiazol-2-yl]-4-(2-hydroxyethoxy)benzamide,
N-[4-(4-chlorothiophen-2-yl)-5-(4-cyclohexylpiperazin-1-yl)thiazol-2-yl]-2-oxo-2,3-dihydrobenzoxazole-6-carboxamide,
3-chloro-N-[4-(4-chlorothiophen-2-yl)-5-(4-cyclohexylpiperazin-1-yl-)thiazol-2-yl]-4-hydroxybenzamide,
(±)-5-chloro-N-[4-(4-chlorothiophen-2-yl)-5-(4-cyclohexylpiperaz-in-1-yl-)thiazol-2-yl]-6-(3-hydroxypyrrolidin-1-yl)nicotinamide,
5-chloro-N-[4-(4-chlorothiophen-2-yl)-5-(4-cyclohexylpiperazin-1-yl-)thiazol-2-yl]-6-(4-hydroxypiperidino)nicotinamide,
5-chloro-N-[4-(4-chlorothiophen-2-yl)-5-(4-propylpiperazin-1-yl-)thiazol-2-yl-]-6-[(2-hydroxyethyl)amino]nicotinamide,
5-chloro-N-[4-(4-chlorothiophen-2-yl)-5-(4-propylpiperazin-1-yl-)thi-azol-2-yl]-6-(4-hydroxypiperidino)nicotinamide,
5-chloro-N-[4-(4-chlorothiophen-2-yl)-5-(4-cyclohexylpiperazin-1-yl-)thiazol-2-yl]-6-(3-oxopiperazin-1-yl)nicotinamide,
6-(4-carbamoylpiperidino)-5-chloro-N-[4-(4-chlorothiophen-2-yl)-5-(-4-cyclohexylpiperazin-1-yl)thiazol-2-yl]nicotinamide,
(±)-5-chloro-N-[4-(4-chlorothiophen-2-yl)-5-(4-cyclohexylpiperaz-in-1-yl)thiazol-2-yl]-6-[(2,3-dihydroxypropyl)amino]nicotinamide,
(±)-5-chloro-N-[4-(4-chlorothiophen-2-yl)-5-(4-cyclohexylpiperaz-in-1-yl)thiazol-2-yl]-6-[(tetrahydro-3-furyl)methoxy]nicotinamide,
6-(4-carbamoylpiperidino)-5-chloro-N-[4-(4-chlorothiophen-2-yl)-5-(-4-propylpiperazin-1-yl)thiazol-2-yl]nicotinamide,
3-chloro-N-[4-(4-chlorothiophen-2-yl)-5-(4-cyclohexylpiperazin-1-yl-)thiazol-2-yl]-4-(4-hydroxypiperidino)benzamide,
1-(2-bromo-4-{[4-(4-chlorothiophen-2-yl)-5-(4-cyclohexylpiperazin-1-yl-)thiazol-2-yl]carbamoyl}phenyl)piperidine-4-carboxylic acid,
1-(2-bromo-4-{[4-(4-chlorothiophen-2-yl)-5-(4-cyclohexylpiperazin-1-yl-)thiazol-2-yl]carbamoyl}phenyl)piperidine-4-carboxamide,
1-(4-{[4-(4-chlorothiophen-2-yl)-5-(4-cyclohexylpiperazin-1-yl)thia-zol-2-yl]carbamoyl-2,6-difluorophenyl)piperidine-4-carboxylic acid,
3-chloro-N-[4-(4-chlorothiophen-2-yl)-5-(4-cyclohexylpiperazin-1-yl-)thiazol-2-yl]-4-(4-cyanopiperidino)benzamide,
1-(4-{[4-(4-chlorothiophen-2-yl)-5-(4-cyclohexylpiperazin-1-yl-)thiazol-2-yl]carbamoyl}-2,6-difluorophenyl)piperidine-4-carboxamide,
3-chloro-N-[4-(4-chlorothiophen-2-yl)-5-(4-propylpiperazin-1-yl)thi-azol-2-yl]-4-(4-hydroxypiperidino)benzamide,
1-(2-chloro-4-{[4-(4-chlorothiophen-2-yl)-5-(4-cyclohexylpiperazin-1-yl-)thiazol-2-yl]carbamoyl}phenyl)piperidine-4-carboxamide,
1-(2-chloro-4-{[4-(4-chlorothiophen-2-yl)-5-(4-propylpiperazin-1-yl-)thiazol-2-yl]carbamoyl}phenyl)piperidin-4-carboxylic acid,
3-chloro-N-[4-(4-chlorothiophen-2-yl)-5-(4-cyclohexylpiperazin-1-yl-)thiazol-2-yl]-4-(4-cyanopiperidino)-5-fluorobenzamide,
1-(2-chloro-4-{[4-(4-chlorothiophen-2-yl)-5-(4-cyclohexylpiperazin-1-yl-)thiazol-2-yl]carbamoyl}-6-fluorophenyl)piperidine-4-carboxamide,
1-(3-chloro-5-{[4-(3-chlorophenyl)-5-(4-cyclohexylpiperazin-1-yl-)thiazol-2-yl]carbamoyl}-2-pyridyl)piperidine-4-carboxylic acid,
5-chloro-N-[4-(4-chlorothiophen-2-yl)-5-(4-cyclohexylpiperazin-1-yl-)thiazol-2-yl]-6-(5-oxo-1,4-diazepan-1-yl)nicotinamide,
[1-(3-chloro-5-{[4-(4-chlorothiophen2-yl)-5-(4-cyclohexylpiperazin-1-yl-)thiazol-2-yl]carbamoyl}-2-pyridyl)piperidin-4-yl]acetic acid,
5-chloro-N-[4-(4-chlorothiophen-2-yl)-5-(4-cyclohexylpiperazin-1-yl-)thiazol-2-yl]-6-{4-[(dimethylamino)carbonyl]piperidino}nicotinamide,
5-chloro-N-[4-(4-chlorothiophen-2-yl)-5-(4-cyclohexylpiperazin-1-yl-)thiazol-2-yl]-6-{4-[(methylamino)carbonyl]piperidino}nicotinamide,
[4-(3-chloro-5-{[4-(4-chlorothiophen-2-yl)-5-(4-cyclohexylpiperazin-1-yl-)thiazol-2-yl]carbmoyl}-2-pyridyl)piperazin-1-yl]acetic acid,
6-[4-(acetylamino)piperidino]-5-chloro-N-[4-(4-chlorothiophen-2-yl)-5-(4-cyclohexylpiperazin-1-yl)thiazol-2-yl]nicotinamide,
3-chloro-N-[4-(4-chlorothiophen-2-yl)-5-(4-cyclohexylpiperazin-1-yl-)thiazol-2-yl]-5-fluoro-4-[4-(methoxyacetyl)piperazin-1-yl]benzamide,
[4-(2-chloro-4-{[4-(4-chlorothiophen-2-yl)-5-(4-cyclohexylpiperazin-1-yl-)thiazol-2-yl]carbamoyl}-6-fluorophenyl)piperazin-1-yl]acetic acid,
3-chloro-N-[4-(4-chlorothiophen-2-yl)-5-(4-cyclohexylpiperazin-1-yl-)thiazol-2-yl]-5-fluoro-4-(4-sulfamoylpiperazin-1-yl)benzamide,
4-[4-(carbamoylmethyl)piperazin-1-yl]-3-chloro-N-[4-(4-chlorothioph-en-2-yl)-5-(4-cyclohexylpiperazin-1-yl)thiazol-2-yl]-5-fluorobenzamide,
5-chloro-N-[4-(4-chlorothiophen-2-yl)-5-(4-cyclohexylpiperazin-1-yl-)thiazol-2-yl]-6-[4-(propylcarbamoyl)piperidino]nicotinamide,
5-chloro-N-[4-(4-chlorothiophen-2-yl)-5-(4-cyclohexylpiperazin-1-yl-)thiazol-2-yl]-6-{4-[(2-ethoxyethyl)carbamoyl]piperidino}nicotinamide, and
pharmaceutically acceptable salts thereof.

Preferably, the compound utilized in the present invention is a compound of Formula X (or a pharmaceutically acceptable salt thereof) having the following structural formula:

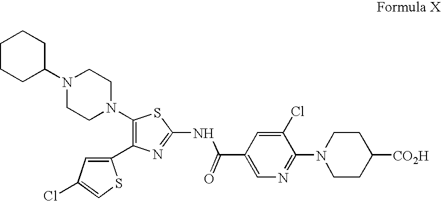

In certain embodiments of the present invention, the compounds of Formula (I)-(V) and (X) may be used as agents for treating or preventing thrombocytopenia by increasing the number of platelets.

In the definition of the general formula for the compound of the present invention, the term “lower” means a straight or branched carbon chain having from 1 to 6 carbon atoms, unless otherwise indicated.

Thus, the “lower alkyl” means alkyls having 1 to 6 carbon atoms, and its examples include methyl, ethyl, propyl, isopropyl, butyl, isobutyl, seobutyl, tert-butyl, pentyl, neopentyl, hexyl, and the like, of which those having 1 to 3 carbon atoms such as methyl, ethyl, propyl, and isopropyl are preferred.

The “lower alkenyl” means alkenyls having 2 to 6 carbon atoms, and its examples include ethenyl, propenyl, butenyl, pentenyl,hexenyl and the like, of which those having 2 to 3 carbon atoms such as ethenyl, 1-propenyl, 2-propenyl, and 3-propenyl are preferred.

The “lower alkylidene” means alkylidenes having 1 to 6 carbon atoms, and its examples include methylidene, ethylidene, propylidene, butylidene, pentylidene, hexylidene, and the like, of which those having 1 to 3 carbon atoms such as methylidene, ethylidene, 1-propylidene, and 2-propylidene are preferred.

The “lower alkylene” means a divalent group of alkyls having 1 to 6 carbon atoms, of which those having 1 to 4 carbon atoms such as methylene, ethylene, trimethylene, methylethylene, tetramethylene, dimethylmethylene, and dimethylethylene are preferred.

The “cycloalkyl” means a carbon ring having 3 to 8 carbon atoms, which may have partial unsaturation. Its examples include cyclopropyl, cyclobutyl, cyclopentyl, cyclohexyl, cyclooctyl, cyclobutenyl, cyclohexenyl, cyclooctadienyl, and the like.

The “aryl” means a mono- to tri-cyclic aromatic ring having 6 to 14 carbon atoms, of which phenyl and naphthyl are preferred, and phenyl is more preferred.

The “arylalkyl” means the “lower alkyl” substituted with the “aryl”, and its examples include benzyl, 1-phenethyl, 2-phenethyl, naphthylmethyl, 1-naphthylethyl, 2-naphthylethyl and the like.

The “monocyclic aromatic heterocycle” means a monovalent group of five- to six-membered aromatic heterocycle or its partially hydrogenated ring, which may comprise a nitrogen, an oxygen, or a sulfur atom, and its examples include thienyl, furyl, pyrrolyl, thiazolyl, oxazolyl, imidazolyl, isothiazolyl, isoxazolyl, pyrazolyl, thiadiazolyl, oxadiazolyl, triazolyl, tetrazolyl, pyridyl, pyrazinyl, pyrimidinyl, pyridazinyl and the like.

The “bicyclic condensed heterocycle” means a monovalent group of an aromatic heterocycle condensed with an aryl or monocyclic aromatic heterocycle, or its partially hydrogenated ring, which may comprise a nitrogen, an oxygen, or a sulfur atom, and its examples include indolyl, isoindolyl, indolizinyl, indazolyl, quinolyl, isoquinolyl, quinolidinyl, phthalazinyl, naphthylidinyl, quinoxalinyl, quinazolinyl, cinnolinyl, benzimidazolyl, imidazopyridyl, benzofuranyl, benzoxazolyl, 1,2-benzoisoxazolyl, benzothienyl, benzothiazolyl, oxazolopyridyl, thiazolopyridyl, indolinyl, isoindolinyl, 1,2-dihydroquinolinyl, 1,2,3,4tetrahydroquinolinyl, 3,4-dihydro-2H-1,4-benzoxazinyl, 1,4-dihydro-2H-3,1-benzoxazinyl, chromanyl, isochromanyl, benzoxolanyl, benzodioxolanyl, benzodioxanyl, and the like.

The “aromatic heterocycle” means the “monocyclic aromatic heterocycle” combined with the “bicyclic condensed heterocycle”.

The “aromatic heterocyclic alkyl” means the “lower alkyl” substituted with the “aromatic heterocycle”, and its examples include thienylmethyl, furylmethyl, pyridylmethyl thiazolylmethyl, oxazolylmethyl, imidazolylmethyl, thienylethyl, furylethyl, pyridylethyl, and the like.

The “non-aromatic heterocycle” means a monovalent group of a non-aromatic heterocycle, which may be condensed with an aryl or monocyclic aromatic heterocycle, and has one or more hetero atoms, which are identical or different, selected from the group consisting of a nitrogen, an oxygen, and a sulfur, and its examples include azetidinyl, pyrrolidinyl, imidazolinyl, imidazolidinyl, pyrazolidinyl, pyrazdinyl, piperidinyl, azepinyl, piperazinyl, homopiperazinyl, morpholinyl, thiomorpholinyl, indolinyl, isoindolinyl, and the like.

The term “halogen” includes fluorine, chlorine, bromine, and iodine atoms.

The “ligand” means a low molecular weight substance binding to an enzyme, receptor, protein, and the like, and includes an agonist and antagonist, of which an agonist is preferred.

As the substituent groups that can be used for the term “optionally substituted” or “which may be substituted”, those commonly used as substituent groups for each group can be used, and each group may have one or more substituent groups.

As the substituent groups that can be used for “aryl or monocyclic aromatic heterocycle, each of which may be substituted” in the definition of R₁, “optionally substituted cycloalkyl”, “optionally substituted aryl”, “optionally substituted arylalkyl”, “optionally substituted aromatic heterocycle”, “optionally substituted aromatic heterocyclic alkyl”, and “optionally substituted nonaromatic heterocycle” in the definitions of R₂₀, R₂₁, R₂₂, R₂₃, R₂₆, R₂₇, R₂₈, and R₂₉, and the “optionally substituted cycloalkyl” and “optionally substituted nonaromatic heterocycle” in the definitions of R₂₄ and R₂₅, the following groups (a) to (h) can be exemplified. Wherein, “R_{Z}” is a lower alkyl which may be substituted with one or more groups selected from the group consisting of —OH, —O-lower alkyl, amino which may be substituted with one or two lower alkyls, carbamoyl which may be substituted with one or two lower alkyls, aryl, aromatic heterocycle, and halogen.
(a) halogen;
(b) —OH, —O—R_{Z}, —O-aryl, —OCO—R_{Z}, oxo(=O);
(c) —SH, —S—R_{Z}, —S-aryl, —SO—R_{Z}, —SO-aryl, SO₂—R_{Z}, —SO₂-aryl, sulfamoyl which may be substituted with one or two R_{Z};
(d) amino which may be substituted with one or two R_{Z}, —NHCO—R_{Z}, —NHCO-aryl, —NHCO₂—R_{Z}, —NHCONH₂, —NHSO₂—R_{Z}, —NHSO₂-aryl, —NHSO₂NH₂, nitro;
(e) —CHO, —CO—R_{Z}, —CO₂H, —CO₂—R_{Z}, carbamoyl which may be optionally substituted with one or two R_{Z}, cyano;
(f) aryl or cycloalkyl, each of which may be substituted with one or more groups selected from the group consisting of —OH, —O-lower alkyl, amino which may be substituted with one or two lower alkyl, halogen and R_{Z};
(g) aromatic heterocycle or nonaromatic heterocycle, each of which may be substituted with one or more groups selected from the group consisting of —OH, —O-lower alkyl, amino which may be substituted with one or two lower alkyl, halogen and R_{Z};
(h) lower alkyl which may be substituted with one or more groups selected from the substituent groups described in (a) to (g).

As the substituent groups that can be used for the “optionally substituted lower alkyl”, “optionally substituted lower alkenyl”, and “optionally substituted lower alkylidene” in the definitions of R₂₀, R₂₁, R|₂₂, R₂₃, R₂₆, R₂₇, R₂₈, and R₂₉, and the “optionally substituted lower alkyl” in the definitions of R₂₄ and R₂₅, the group described in (a) to (g) can be exemplified.

As the substituent groups that can be used for the “aryl, monocyclic aromatic heterocycle, or bicyclic condensed heterocycle, each of which may be substituted” in the definition of Ar₁, oxo (with the proviso that oxo can be used only for a bicyclic condensed heterocycle); and a group represented by the general Formula (VI) can be exemplified.

-A-B—C-D-E

wherein A is a single bond, or optionally substituted cyclic aminediyl (with the proviso that the cyclic aminediyl is bound to Ar₁ with nitrogen atom of the cyclic amine thereof.); B is a single bond, —O—, —NH—, —N(—R_{Z})—, —NHCO—, —CO—, —CONH—, or —CON(—R_{Z}); C is a single bond; or, lower alkylene or lower alkenylene, each of which may be substituted with one or more groups selected from the group consisting of halogen and —OH; D is a single bond, —NHCO—, —NHSO₂—, —CO—, or —SO₂—; E is a H; halogen; —OH; —O—R_{Z}; —O—CO—R_{Z}; amino which may be substituted with one or two R_{Z}; —R_{Z}; cyano; aryl, cycloalkyl, aromatic heterocycle or nonaromatic heterocycle, each of which may be substituted, with the proviso that —CH₂-nonaromatic heterocycle, and —CH═CH-nonaromatic heterocycle (with the proviso that the carbon atom of the nonaromatic heterocycle is substituted with methyne) are excluded from the group represented by the general Formula (VI); and in case Ar₁ is an aryl or monocyclic aromatic heterocycle, each of which may be substituted, the following groups are excluded:

a group wherein -A- and —B— form a single bond, —C— is a single bond, or ethylene or vinylene, each of which may be substituted with one or more groups selected from the group consisting of halogen and —OH, and -D- is —CO—,

a group wherein -A- and —B— form a single bond, —C— is a single bond, or ethylene or vinylene, each of which may be substituted with one or more groups selected from the group consisting of halogen and —OH, -D- is —SO₂—, and -E- is amino which may be substituted with one or two R_{Z},

a group wherein -A- and —B— form a single bond, —C— is a single bond, or ethylene or vinylene, each of which may be substituted with one or more groups selected from the group consisting of halogen and —OH, -D- is a single bond -E- is a monovalent group of aryl, partially unhydrogenated monocyclic aromatic heterocycle, or a ring condensed with partially unhydrogenated monocyclic aromatic heterocycle, each of which may be substituted,

a group wherein -A- is a single bond, and —B— is —CO—,

a group wherein -A-, —B—, —C— and -D- form a single bond, and -E- is a monovalent group of aryl, partially unhydrogenated monocyclic aromatic heterocycle, or a ring condensed with partially unhydrogenated monocyclic aromatic heterocycle.

The “cyclic aminediyl (with the proviso that the cyclic aminediyl is bound to Ar₁ with nitrogen atom of the cyclic amine thereof.)” in the definition of -A- means a divalent group of three to eight-membered (in the case of a condensed ring or spiro ring, five- to fifteen-membered) aromatic or nonaromatic cyclic amines, which have at least one nitrogen atom, and may have one or more hetero atoms, identical or different, selected from the group consisting of nitrogen, oxygen, and sulfur, including a condensed ring and spiro ring, and Ar₁ is directly substituted with the at least one nitrogen atom. Its examples include divalent groups of azepine, pyrrolidine, piperidine, piperazine, N-methylpiperazine, azepane, diazepane, N-methyldiazepane, morpholine, thiomorpholine, isoindoline, 1,4-dioxa-8-azaspiro[4,5]decane, 1-oxa-8-azaspiro-[4,5]decane, 1-oxa-8-azaspiro[4,5]undecane, and the like.

As the substituent groups that can be used for the “optionally substituted cyclic aminediyl” in the definition of -A- and the “aryl, cycloalkyl, aromatic heterocycle, or nonaromatic heterocycle, each of which may be substituted” in the definition of -E-, the groups described in (a) to (h), and lower alkylidene which may be substituted with the groups (a) to (h) can be exemplified.

The human TPO receptor agonists of the present invention represented by the general Formula (I) or (V) may comprise asymmetric carbon atoms depending on the kinds of substituent groups. In certain embodiments, optical isomers may exist based on the asymmetric carbon atom. The human TPO receptor agonists of the present invention may include a mixture of these optical isomers or isolated ones. In certain embodiments, tautomers of the human TPO receptor agonists of the present invention may exist (the tautomer, 2-hydroxypyridine and 2-pyridone can be exemplified). In yet another embodiment, the human TPO receptor agonists of the present invention may include isomers as a mixture or as an isolated isomer. In certain embodiments, the human TPO receptor agonists are labeled compounds, i.e., compounds wherein one or more atoms are labeled with radioisotopes or non-radioisotopes, are also included in the present invention.

The human TPO receptor agonists utilized in the present invention may be in the form of the free base, a pharmaceutically acceptable salt, polymorph, metabolite, derivative or any combination of the foregoing.

Pharmaceutically acceptable salts may include, but are not limited to, mineral acids such as hydrochloric acid, hydrobromic acid, hydroiodic acid, sulfuric acid, nitric acid, phosphoric acid, and the like; an organic acid such as formic acid, acetic acid, propionic acid, oxalic acid, malonic acid, succinic acid, fumaric acid, maleic acid, lactic acid, malic acid, tartaric acid, citric acid, methanesulfonic acid, ethanesulfonic acid, p-toluenesulfonic acid, aspartic acid, glutamic acid, and the like; salts with an inorganic base such as sodium, potassium, magnesium, calcium, and the like; salts with an organic base such as methylamine, ethylamine, ethanolamine, lysine, ornithine, and the like; and ammonium salts, and the like.

In certain other embodiments, the human TPO receptor agonists may be in the form of a hydrate or a solvate. In another embodiment, the human TPO receptor agonists may include a prod-drug that is metabolized to one or more of the human TPO receptor agonists of the general Formula (I) or (V).

The agent(s) utilized in the present invention may be contained in an oral dosage form together with a pharmaceutically acceptable ingredient.

Pharmaceutically acceptable ingredients may include, but are not limited to acidifying agents, antimicrobial agents, alkalinizing agents, antioxidants, antiseptic agents, bacteriostatic agents, binders, buffering agents, coating agents, desiccants, diluents, dispersing agents, emollients, emulsifying agents, fillers, film-formers, flavoring agents, gelling agents, granulating agents, lubricants, plasticizers, preservatives, solubilizing agents, stiffening agents, suspending agents, sweetening agents, viscosity increasing agents, wetting agents, and the like.

In certain preferred embodiments, the agent(s) may be contained in an immediate release oral dosage form. In other preferred embodiments, the agent(s) may be contained in a controlled-release dosage form. The dosage forms of the present invention may include, but are not limited to tablets and soft or hard gelatin capsules.

In certain embodiments, agent(s) may be contained within multiparticulates that can be compressed into tablets or filled into a soft or hard gelatin capsules. The multiparticulates may be spheroids, beads, pellets, rods, microparticles, e.g., microspheres, and the like.

In certain embodiments, the oral dosage form may be a controlled-release dosage form, wherein the agent(s) are contained in a controlled-release matrix. In other embodiments, the agent(s) may be contained within controlled-release multiparticulates. In yet another embodiment, the agent(s) may be contained in an immediate release dosage form that has a controlled-release coating.

The multiparticulates of the present invention may be compressed in to tablets or filled into soft or hard gelatin capsules to provide for an oral solid dosage form. In certain embodiments, the tablets and capsules of the present invention may be coated with an immediate release coating, a controlled-release coating or an enteric coating.

In another embodiment, the agent(s) may be coated onto beads to provide immediate release beads. In certain embodiments, the beads may be coated with an immediate release coating. In other embodiments, the beads may be coated with a controlled-release coating.

In certain other embodiments, the agent(s) may be contained in an oral solution, emulsion, suspension, and the like.

While oral dosage forms are preferred, it is contemplated that the agent(s) may be administered parenterally as an injection or nasogastrically as a solution, suspension, an emulsion and the like.

When combination therapy is contemplated, the agents may be administered in the same or different dosage forms, and by the same or by different routes of administration.

### DETAILED DESCRIPTION OF THE PREFERRED EMBODIMENTS

The following examples illustrate various aspects of the invention. They are not to be construed to limit the claims in any manner whatsoever.

### Example I

Thrombopoietin (TPO) is a 332 amino acid cytokine that is the principal physiologic regulator of platelet production. Using a high-throughput growth assay based on the proliferation of human receptor (c-Mp1)-expressing Ba/F3 cells (c-Mp1-Ba/F3 cells), the screening of a library led to the identification of a novel series of c-Mp1 agonists. Modification of the structure resulted in the discovery of FORMULA X, which displayed efficacies equivalent to those of TPO in several cell-based assays, such as proliferation in a c-Mp1-dependent manner (EC50=3.3 nM, no effect on Ba/F3 cell growth) as well as the induction of megakaryocyte colony formation of human cord blood CD34+ cells (EC50=25 nM).

When G-CSF-mobilized human peripheral blood CD34+ cells were incubated with rhTPO or FORMULA X for periods of 12 days, and examined for the degree of polyploidy, it was found that the ploidy level of cells treated with FORMULA X was not different from that of cells treated with rhTPO. Importantly, FORMULA X treatment elicited signal-transduction responses, such as STAT3, STAT5, and ERK activation, in human c-Mp1-expressing Ba/F3 cells similar to those by rhTPO treatment. In this respect, FORMULA X has demonstrated remarkable species specificity for TPO receptor agonist activity, as the activation of signaling pathways occurred only in human and chimpanzee platelets.

Platelets of other species, e.g., baboons, rhesus monkeys, cynomolgus monkeys, squirrel monkeys, common marmosets, beagle dogs, guinea pigs, pigs, rabbits, hamsters, rats, or mice, showed no signaling responses to FORMULA X treatment as judged by anti-phospho-STAT5 immunoblot assays, while platelets of all these species showed signaling responses to rhTPO treatment.

Using human platelets, effect of FORMULA X on TPO binding to human c-Mp1 was also examined, and it was found that FORMULA X did not influence rhTPO binding to human c-Mp1 at concentrations up to 100 microM. These findings thus demonstrate that the site of action of FORMULA X on human c-Mp1 is distinct from that of rhTPO. Our data thus suggest that FORMULA X is a novel TPO receptor agonist acting specifically on human platelets, and that it should be a useful agent for treatment of thrombocytopenia in man.

### Example II

### Effect of Formula X with TPO on the Differentiation of Human CD34⁺ Cells into Megakaryocytes

In this study, the effect of FORMULA X in combination with TPO on megakaryocytopoiesis was examined.

G-SCF-mobilized human peripheral blood CD34+ cells were cultured with a combination of FORMULA X and TPO, FORMULA X, or rhTPO in a serum-free liquid culture system. The numbers of CD34+CD41− cells (hematopoietic progenitor cells), CD34+CD41+ cells (megakaryocytic progenitor cells), and CD34−CD41+ cells (megakaryocytes) were measured using flow cytometry.

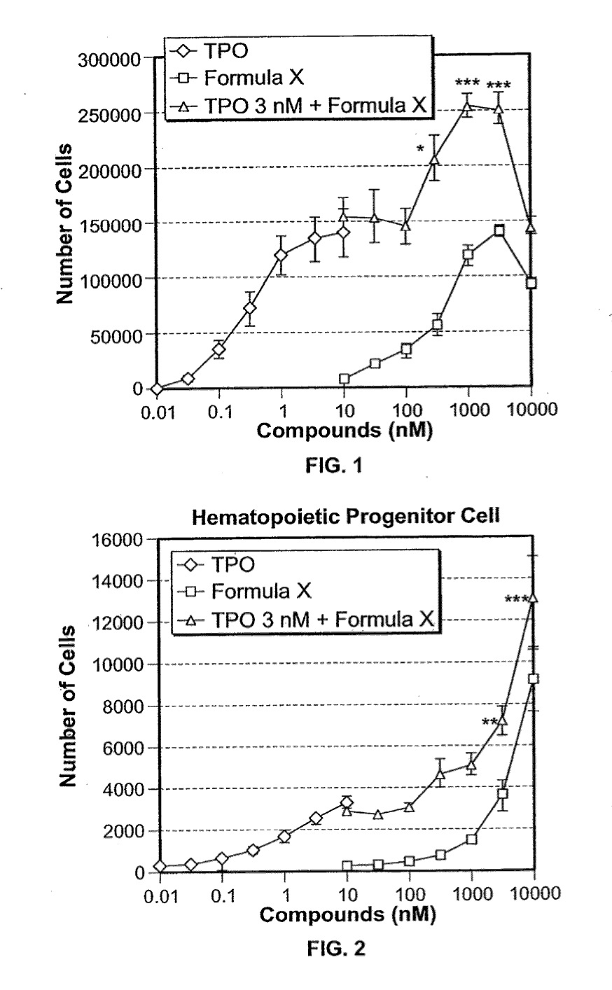

On day 14, FORMULA X or TPO alone increased the number of megakaryocytes in a dose-dependent fashion, and the maximum activity of FORMULA X was similar to that of TPO (FIG. 1). Furthermore, FORMULA X dose-dependently increased the number megakaryocytes in the presence of 3 nM TPO, the maximum effect on megakaryocyte differentiation (FIG. 1).

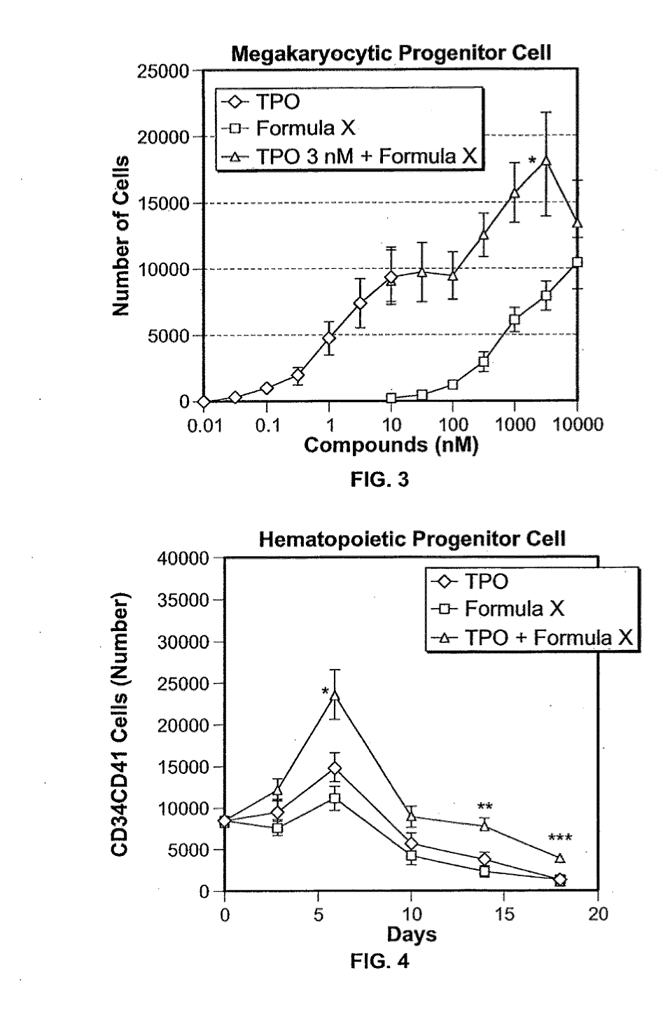

The use of FORMULA X in combination with TPO was supposed to have an additive effect on megakaryopoiesis. The number of hematopoietic progenitor cells (FIG. 2) and megakaryocytic progenitor cells (FIG. 3) were increased to a greater degree by a combination of FORMULA X and TPO compared with FORMULA X or TPO only. These results showed that FORMULA X, in combination with TPO, expanded not only megakaryocytes, but also hematopoietic progenitor cells and megakaryocytic progenitor cells.

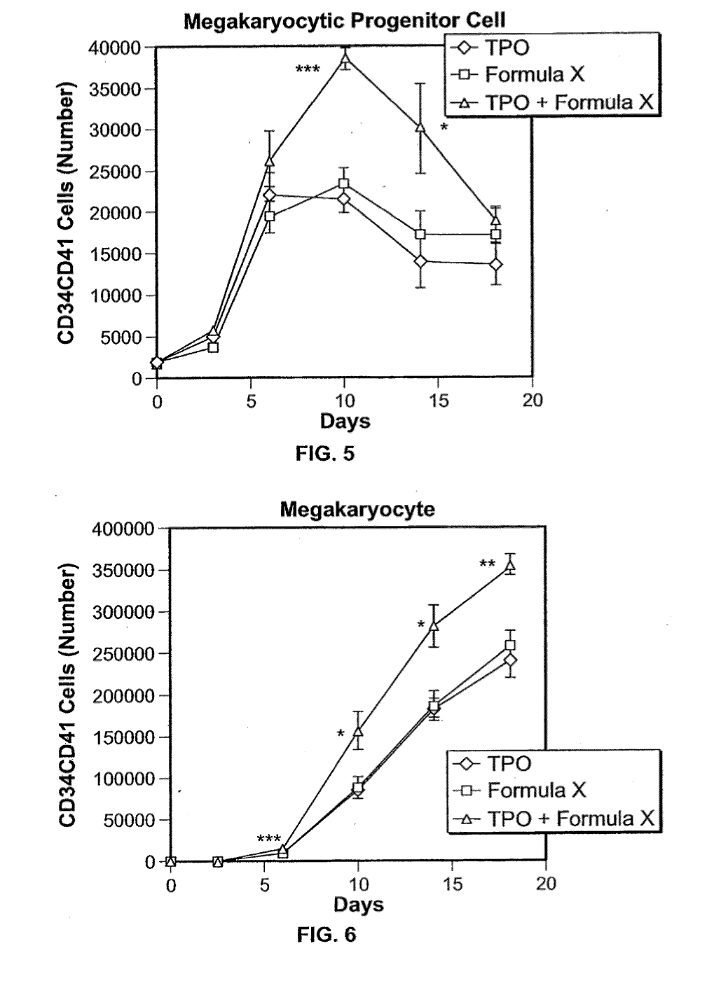

Next, the time course of changes in the numbers of each type cell were evaluated in the presence of 3 μM FORMULA X, 3 nM TPO, or 3 μM FORMULA X+3nM TPO (FIGS. 4, 5 and 6). Compared with FORMULA X or TPO only, the combination of FORMULA X and TPO augmented the number of hematopoietic progenitor cells at and after day 6 (FIG. 4), the number of megakaryocytic progenitor cells at day 10 and day 14 (FIG. 5), and the number of megakaryocytes at and after day 6 (FIG. 6). These results suggested that the additive effect of FORMULA X and TPO to a greater degree served to increase the number of hematopoietic progenitor cells in the early stages of culturing.

For purposes of this study, the number of megakaryocytes (CD34−CD41+ cells) depicted in FIG. 1 were counted using flow cytometry. The data represents the mean±SE of 5 independent experiments. *: p<0.05, ***p<0.001 compared with 3 nM TPO using Dunnett's test.

The number of hematopoietic progenitor cells (CD34−CD41+ cells: a) and megakaryocytic progenitor cells (CD34+CD41+ cells: b) depicted in FIGS. 2 and 3, respectively, were counted using flow cytometry, then compared with 3 nM TPO using Dunnett's test.

On the indicated days, the numbers of hematopoietic progenitor cells (CD34−CD41+ cells: a), megakaryocytic progenitor cells (CD34+CD41+ cells: b), and megakaryocytes (CD34−CD41+ cells: c) depicted in FIGS. 4, 5, and 6 were counted using flow cytometry on indicated days. The data represent the mean±SE of 5 independent experiments. *:p<0.05, **p<0.01, ***p<0.001 compared with 3 nM TPO using Dunnett's tests.

### Example III

Non-obese diabetic/severe combined immunodeficiency (NOD/SCID) mice were characterized as an efficient engraftment model for human hematopoietic stem cells, as this model results in the production of human platelets. In this way, we examined the in vivo platelet-increasing effect of FORMULA X in human platelet-producing NOD/SCID mice in which human hematopoietic stem cells were transplanted.

In this study, we used commercially available cryopreserved human fetal liver CD34+ cells as a source of human hematopoietic stem cells. The cells were transplanted into sublethally irradiated (240 cGy) NOD/SCID mice. Human platelets started to appear in peripheral blood of these mice 4 weeks after transplantation. The production of human platelets continued up to six months post-transplant. Various doses of FORMULA X (0, 0.3, and 3 mg/kg/day) were orally administered for 14 days to NOD/SCID mice that had been confirmed to produce human platelets stably.

Oral administration of FORMULA X dose-dependently increased the number of human platelets produced by these mice, with significance at 1 mg/kg/day and above. The increase in the human platelet count reached about 2.7-fold at 1 mg/kg/day and about 3.0-fold at 3 mg/kg/day on day 14. Withdrawal of FORMULA X administration caused the human platelet count to return to the pretreatment level. The number of murine platelets did not change during the study period.

Next, to evaluate the function of human platelets produced in peripheral blood of these mice, the expression of activation-dependent marker CD62P (P-selectin) on human platelets stimulated with thrombin receptor agonist peptide (TRAP) were examined. CD62P expression on human platelets was induced by the stimulation of blood from transplanted mice with TRAP, suggesting that human platelets produced in NOD/SCID mice were functional. Furthermore, the maximum response of CD62P expression on human platelets induced by TRAP was evaluated before and after administration of FORMULA X, which was similar to the results obtained with a vehicle group. These results suggest that FORMULA X is an orally active TPO receptor agonist useful for treating patients with thrombocytopenia.

In the preceding specification, the invention has been described with reference to specific exemplary embodiments and examples thereof. It will, however, be evident that various modifications and changes may be made thereto without departing from the broader spirit and scope of the invention as set forth in the claims that follow. The specification and drawings are accordingly to be regarded in an illustrative manner rather than a restrictive sense.

1-37. (canceled)

38. A method for treating thrombocytopenia, the method comprising:
co-administering to a patient in need thereof a compound of Formula I: 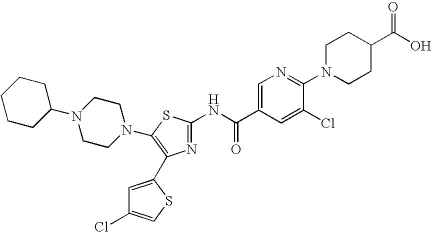
or pharmaceutically acceptable salts thereof, and
a second pharmaceutical agent selected from the group consisting of rThrombopoietin, AMG-531, GSK-115, analgesic agents, antihemophilic factor, antihemorrhagic agents, antineoplastic agents, antiulcerative agents, antacids, corticosteroids, growth hormones, hematinic agents, immunosupressants, platelet-activating factors and any combinations and mixtures thereof.

39. The method of claim 38, wherein the patient is already being treated with the second agent.

40. The method of claim 39, wherein the compound of Formula I and second agent are administered in different dosage forms.

41. The method according to claim 38, wherein the compound of Formula I and the second agent are administered in the same dosage form.

42. The method of claim 38, wherein the compound of Formula I and the second agent are administered simultaneously.

43. The method of claim 38, wherein the compound of Formula I and the second agent are administered sequentially.

44. The method of claim 40, wherein the compound of Formula I and the second agent are administered by the same route of administration.

45. The method of claim 38, wherein the compound of Formula I and the second agent are administered by different routes of administration.

46. The method of claim 38, wherein the co-administration achieves expanded megakaryocytes, hematopoietic progenitor cells and megakaryocytic progenitor cells.

47. The method of claim 38, wherein the compound of Formula I is administered in a controlled release tablet or capsule.

48. The method of claim 38, wherein the compound of Formula I is administered in an immediate release tablet or capsule.

49. The method of claim 38, wherein the thrombocytopenia is selected from idiopathic thrombocytopenic purpura, disease inherent thrombocytopenia, thrombocytopenia from a preexisting medical condition, or thrombocytopenia induced by chemotherapy.

50. The method of claim 38, wherein the compound of Formula I is administered in a dose of from about 0.01 mg/kg/day to about 10 mg/kg/day.

51. The method of claim 50, wherein the compound of Formula I is administered in a dose of from about 0.01 mg/kg/day to about 3 mg/kg/day.

52. The method of claim 51, wherein the compound of Formula I is administered in a dose of from about 0.5 mg/kg/day to about 3 mg/kg/day.

53. The method of claim 50, wherein the compound of Formula I is administered in a dose of from about 0.1 mg/kg/day to about 2 mg/kg/day.

54. The method of claim 50, wherein the compound of Formula I is administered in a dose of from about 1 mg/kg/day to about 3 mg/kg/day.

55. The method of claim 38, wherein the first agent is rhTPO.

56. A method for treating thrombocytopenia in a subject in need of a transfusate, the method comprising:
co-administering to a patient in need thereof a compound of Formula I: 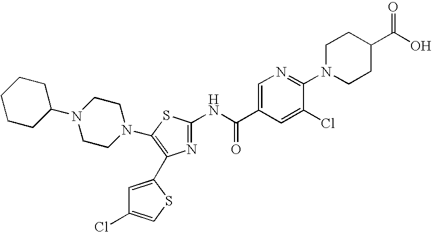
or pharmaceutically acceptable salts thereof, and
a tranfusate.

57. The method of claim 56, wherein administration of the compound of Formula I increases platelets as compared to administration of the transfusate alone.
